(19) United States
(12) Patent Application Publication
(10) Pub. No.: US 2013/0121921 A1
(43) Pub. Date: May 16, 2013

(54) Rodent Therapeutic Model And Methods

(71) Applicant: The Christus Stehlin Foundation For Cancer Research, Houston, TX (US)

(72) Inventor: Beppino GIOVANELLA, Houston, TX (US)

(73) Assignee: The Christus Stehlin Foundation For Cancer Research, Houston, TX (US)

(21) Appl. No.: 13/673,190

(22) Filed: Nov. 9, 2012

Related U.S. Application Data
(60) Provisional application No. 61/558,665, filed on Nov. 11, 2011.

(51) Int. Cl. A01K 67/027

(52) U.S. Cl. CPC A01K 67/0271
USPC 424/9.2; 800/10

(57) ABSTRACT

Rodent therapeutic models are provided containing at least human hepatocytes and at least one of human hematopoietic bone marrow cells and human cord blood. Such rodent therapeutic models can also be immunodeficient and/or contain human intestinal enzymes. These partially humanized rodent therapeutic models can have at least partially humanized bone marrow and a partially humanized liver. These rodent models include mice. The rodent models can be transplanted with one or more tumors, for example, xenotransplantation with a human tumor. Toxicological and efficacy trials of various therapies, for example, anti-cancer therapies, can be performed with these rodent therapeutic models. Oral administration of camptothecin can demonstrate increased toxicity in the humanized mice.

This application claims the benefit under 35 U.S.C. §119(e) of prior U.S. Provisional Patent Application No. 61/558,665, filed Nov. 11, 2011, which is incorporated in its entirety by reference herein.

### BACKGROUND OF THE INVENTION

The present invention relates to a humanized rodent model for studying toxicity and efficacy of anti-cancer therapeutics and/or other therapeutics.

Humanization of the mouse bone marrow and hematopoietic organs has been mostly pursued by immunologists interested in developing a human immune system outside the human body (Ishikawa et al., Blood, 106 (5):1565-1573, 2005; Marodon et al., Eur. J. Immunol., 39 (8):2136-2145, 2009). The humanization of the mouse liver has been the province of virologists working to develop a model for the treatment of hepatitis (Turrini et al., Transplant Proceedings, 38 (4):1181-1184, 2006; Washburn et al., Gastroenterology, 140 (4):1334-1344, 2010) and of pharmacologists interested in the drugs metabolism (Strom et al., Methods Mol. Biol., 640:491-509, 2010; Kaminura et al., Drug Metab. Pharmacokinet., 225 (3):223-35, 2010). Hematologists have been interested in the development of very immunosuppressed mice, because while the nude mouse accepts xenotransplants of human solid tumors, leukemia and lymphomas (with the exception of Burkitt lymphomas) cannot be grown as xenotransplants and many implants in privileged sites also do not grow well.

The BLT Mouse (Wege et al., Curr. Top. Microbiol. Immunol., 324:149-165; Lan et al., Blood, 108 (2):487-492, 2006; Brainard et al., J. Virol., 83 (14):7305-7321., 2009; Denton et al., J Virol., 85 (15):7582-7593, 2011) is produced by co-transplanting human fetal liver (in the fetus the liver is a hematopoietic organ) and thymus tissues plus CD34⁺ fetal liver cells in NOD/SCID or NSG mice that have been pre-irradiated (2 Gy). Fragments of thymus and liver are implanted under the renal capsule and CD34⁺⁺ 1-5×10⁶ cells are injected intravenously. These mice are quite well reconstituted as far as the hematopoietic system is concerned but have a mouse liver and metabolism.

One of the main systems currently used to assess the potential of anticancer agents is the nude mouse xenograft system. Certain limitations of that system make extrapolating data generated through it a somewhat inaccurate process. Specifically, while the target of the chemotherapy is human (tumor xenograft lines), the sites drug metabolism (usually the liver) and primary drug toxicity (bone marrow) will not accurately reflect what will happen in a human patient because of differences between human and murine physiology. Work with camptothecins has demonstrated the limitations of the current xenograft methodology. In nude mice, there is essentially no xenograft tumor line that cannot be eradicated. In human patients, however, at best, only a 20-30% response, almost all of which were partial, was found. After more than 40 years of work and over 200,000 mice used, xenografts in the nude mouse are not as accurate a predicting system for human chemotherapy as could be hoped for. Accordingly, a need exists for better therapeutic models for testing human therapies.

### SUMMARY OF THE PRESENT INVENTION

It is therefore a feature of the present invention to provide a rodent therapeutic model that is at least partially humanized as to bone marrow and the liver.

Another feature of the present invention is to provide a rodent therapeutic model that is also at least partially humanized as to intestinal enzymes.

A further feature of the present invention is to test the toxicity and efficacy of various therapies using such rodent therapeutic models.

Additional features and advantages of the present invention will be set forth in part in the description that follows, and in part will be apparent from the description, or may be learned by practice of the present invention. The objectives and other advantages of the present invention will be realized and attained by means of the elements and combinations particularly pointed out in the description and appended claims.

To achieve these and other advantages, and in accordance with the purposes of the present invention, as embodied and broadly described herein, the present invention relates to a rodent containing at least human hepatocytes and at least one of human hematopoietic bone marrow cells and human cord blood. The human hepatocytes, the human hematopoietic bone marrow cells, the human cord blood, or any combination thereof can comprise CD34⁺ cells. The rodent can further contain humanized and/or human intestinal enzymes.

The present invention further relates to a method of producing a rodent containing at least human hepatocytes and at least one of human hematopoietic bone marrow cells and human cord blood. The human hepatocytes, the human hematopoietic bone marrow cells, the human cord blood, or any combination thereof can comprise CD34⁺ cells. A rodent can be prepared for bone marrow engraftment. Human hematopoietic bone marrow cells are transplanted into the rodent. Alternatively, or in addition, human cord blood can be transplanted, e.g., transfused, into the rodent. Human hepatocytes can be transplanted into the rodent. The method can produce a rodent fully or partially humanized for bone marrow and fully or partially humanized for liver.

The present invention also relates to a method of testing a toxicity of a therapy. A therapy is administered to a rodent by any suitable means. The rodent contains at least human hepatocytes and at least one of human hematopoietic bone marrow cells and human cord blood. The human hepatocytes, the human hematopoietic bone marrow cells, the human cord blood, or any combination thereof can comprise CD34⁺ cells. The rodent can have at least partially humanized bone marrow and at least a partially humanized liver. At least one toxicity metric is measured in the rodent. The toxicity of the therapy is determined based on the at least one toxicity metric measured. The toxicity is assigned based on the determination.

The present invention still further relates to a method of testing the efficacy of a therapy. A therapy is administered to a rodent, the rodent containing at least human hepatocytes and at least one of human hematopoietic bone marrow cells and human cord blood. The human hepatocytes, the human hematopoietic bone marrow cells, the human cord blood, or any combination thereof can comprise CD34⁺ cells. At least one efficacy metric in the rodent is measured. The efficacy of the therapy is determined based on the at least one efficacy metric measured. The efficacy of the therapy is assigned.

It is to be understood that both the foregoing general description and the following detailed description are exemplary and explanatory only and are intended to provide a further explanation of the present invention, as claimed.

### DETAILED DESCRIPTION OF THE PRESENT INVENTION

A rodent containing at least human hepatocytes and at least one of human hematopoietic bone marrow cells and human cord blood is provided according to the present invention. The human hepatocytes, the human hematopoietic bone marrow cells, the human cord blood, or any combination thereof can comprise CD34⁺ cells. Unless otherwise specified, any reference to CD34⁺ cells can include CD34^{+/+} cells, CD34^{+/+} cells, or any combination thereof. The rodent can contain partially humanized bone marrow and a partially humanized liver. The rodent can contain a fully humanized bone marrow and a partially humanized liver. The rodent can contain a fully humanized liver. Humanized rodent bone marrow includes at least some human bone marrow cells, for example, hematopoietic bone marrow cells. The hematopoietic bone marrow cells can be derived from human cord blood. Humanized rodent bone marrow can also contain non-hematopoietic human bone marrow cells. A humanized rodent liver includes at least some human hepatocytes. The rodent can contain an artificial liver containing human hepatocytes. The rodent can have a partial or full hepatectomy leaving part or none of the rodent liver. At least one tenth, at least one fifth, at least one quarter, at least one third, at least one half, at least two thirds, at least three quarters, at least four fifths, or at least nine tenths of the rodent liver can be removed or retained. The rodent can further contain humanized and/or human intestinal enzymes.

The rodent containing at least human hepatocytes and at least one of human hematopoietic bone marrow cells and human cord blood can also be immunodeficient. Immunodeficiency can be achieved by one or more genetic mutation, chemical treatment, radiation treatment, or a combination thereof. The rodent can be severely combined immunodeficient (SCID). The rodent can be male or female. The rodent can be any age. The rodent can be a fetus. The rodent can be less than one week old, from about one week to about five years old, from about one week to about three years old, from about two weeks to about two years old, from about three weeks to about one year old, from about four weeks to about six months old, from about six weeks to about three months old, from about eight weeks to about ten weeks old, older than three years old, or older than five years old.

The rodent can be any species, subspecies, genetic variant, tissue variant, or combination thereof, of rodent. Examples of rodent species include rat, mouse, hamster, and gerbil.

The present invention is not limited to rodents and is also applicable to other mammals and non-mammals. The mouse can be an immunodeficient mouse. The mouse can be a nude mouse. The mouse can be a severely combined immunodeficient (SCID) mouse, for example, a NOD/SCID/1L2ZY null (NSG) mouse. The NSG mouse is described in Pearson et al., Curr. Top. Microbiol. Immunol., 324:25-51, 2008; Shultz et al., Curr Top Microbiol Immunol., 324:25-51, 2005; Strom et al., Methods Mol. Biol., 640:491-509, 2010; McDermott et al., Blood., 116 (2):193-200, 2010; Lepus et al., Hum. Immunol., 70 (10):790-802, 2009; Brehm et al., Clin Immunol., 135 (1):84-98, 2010. Any suitable immunodeficient rodent can be used. Suitable rodents can be obtained from such sources as The Jackson Laboratory of Bar Harbor, Me., Charles River Laboratories International, Inc. of Wilmington, Mass., and Harlan Laboratories of Indianapolis, Ind.

The rodent of the present invention can contain one or more human neoplastic cells. Any type, number, or combination of human neoplastic cells can be present in the rodent. Neoplastic cells can be benign or malignant. Neoplastic cells can be metastatic. The rodent can contain any type, number, or combination of human tumors and/or cancers. The human tumor or other cancer morphology can include a human carcinoma, a human sarcoma, a human lymphoma, or a combination thereof. The tumor or cancer can be, for example, breast cancer, prostate cancer, lung cancer, colon cancer, rectal cancer, urinary bladder cancer, non-Hodgkin lymphoma, melanoma, renal cancer, pancreatic cancer, cancer of the oral cavity, pharynx cancer, ovarian cancer, thyroid cancer, stomach cancer, brain cancer, multiple myeloma, esophageal cancer, liver cancer, cervical cancer, larynx cancer, cancer of the intrahepatic bile duct, acute myeloid leukemia, soft tissue cancer, small intestine cancer, testicular cancer, chronic lymphocytic leukemia, Hodgkin lymphoma, chronic myeloid cancer, acute lymphocytic cancer, cancer of the anus, anal canal, or anorectum, cancer of the vulva or cancer of the neck, gall bladder, or pleura, malignant mesothelioma, bone cancer, cancer of the joints, hypopharynx cancer, cancer of the eye, cancer of the nose, nasal cavity, neck, or middle ear, nasopharynx cancer, ureter cancer, peritoneum, omentum, or mesentery cancer, or gastrointestinal carcinoid tumor.

A method of producing a rodent containing at least human hepatocytes and at least one of human hematopoietic bone marrow cells and human cord blood is provided by the present invention. The human hepatocytes, the human hematopoietic bone marrow cells, the human cord blood, or any combination thereof can comprise CD34⁺ cells. A rodent can be prepared for bone marrow engraftment and/or human cord blood transplantation, e.g., transfusion. At least one of human hematopoietic bone marrow cells are transplanted into or human cord blood is transplanted, e.g., transfused, into the rodent. Human hepatocytes can be transplanted into the rodent. The method can produce a rodent fully or partially humanized for bone marrow and fully or partially humanized for liver.

The preparation of the rodent for bone marrow engraftment and/or human cord blood transplantation, e.g., transfusion, can include administering busulfan or a pharmaceutically acceptable salt thereof, or an equivalent compound, to the rodent. Busulfan and/or an equivalent compound can be supplemented with cyclophosphamide, total body irradiation, or a combination thereof The transplanting of human bone marrow cells can include transplanting human hematopoietic bone marrow cells enriched in human stem cells. Stem cells can include embryonic stem cells, adult stem cells, umbilical cord-derived stem cells, or a combination thereof Human hematopoietic bone marrow cells can be transplanted using any suitable means, for example, human hematopoietic bone marrow cells can be injected into the rodent. Non-hematopoietic human bone marrow cells can also be transplanted. Human hepatocytes can be transplanted into the rodent by any suitable means. The transplanting of human hepatocytes can involve transplanting an artificial liver containing human hepatocytes into the peritoneal cavity of the rodent. The transplanting of human hepatocytes can include inoculating human hepatocytes into the spleen of the rodent. The method can include performing a partial or full hepatectomy on the rodent liver. Once the animals are stably engrafted with human bone marrow (e.g., 2-3 mos. after injection) the mice can receive an artificial liver constructed as described in Chen et al., Proc. Natl. Acad. Sci., 108 (29):11842-11847, 2011. That procedure can be modified by adding a partial hepatectomy (⅔) of the mouse liver and the intransplenic inoculation of 2×10⁶ human hepatocytes.

The method can include transplanting a human neoplastic cell into the rodent. Any number, type, or combination of human neoplastic cells can be transplanted into the rodent. The method can include transplanting any number, type, or combination of human tumors into the rodent. The human tumor can contain a human carcinoma, a human sarcoma, a human lymphoma, or a combination thereof. Transplantation of a human neoplastic cell or human tumor can be performed before, during, or after the transplantation of the human hematopoietic bone marrow cells, transplantation, e.g., transfusion, of human cord blood, and/or transplantation of human hepatocytes.

The method can be performed on a male rodent, a female rodent, or both. The rodent can be any rodent species, subspecies, genetic variant, tissue variant, or combination thereof, of rodent. The method is also applicable for non-rodent mammalian species and non-mammalian species. The method can be performed on an immunodeficient rodent. The rodent can be a mouse. The mouse can be a NOD/SCID/1L2ZY null (NSG) mouse.

The method can further include transplanting a human intestinal enzyme, a cell expressing a human intestinal enzyme, genetic material encoding a human intestinal enzyme, or a combination thereof, into the rodent. Any number, type, or combination of human intestinal enzymes can be transplanted. A human intestinal enzyme can be performed before, during, or after the transplanting of the human hematopoietic bone marrow cells, human cord blood, and/or human hepatocytes. A mouse having humanized intestinal enzymes can be constructed, for example, as described in van Herwaarden et al., J. Clin. Invest., 117 (11):3583-3592, 2007.

A method of testing a toxicity of a therapy is provided by the present invention. A therapy is administered to a rodent by any suitable means. The rodent contains at least human hepatocytes and at least one of human hematopoietic bone marrow cells and human cord blood. The human hepatocytes, the human hematopoietic bone marrow cells, the human cord blood, or any combination thereof can comprise CD34⁺ cells. The rodent has at least partially humanized bone marrow and at least a partially humanized liver. At least one toxicity metric is measured in the rodent. The toxicity of the therapy is determined based on the at least one toxicity metric measured. The toxicity is assigned based on the determination.

The measuring of the method can include measuring a biomarker level in the rodent. The measuring can further include measuring the biomarker level in a rodent that is not administered the therapy, and the determining can include comparing the measured biomarker level in the administered-to rodent relative to the measured biomarker level in the non-administered-to rodent. The non-administered-to rodent can also contain at least human hepatocytes and at least one of human hematopoietic bone marrow cells and human cord blood. The human hepatocytes, the human hematopoietic bone marrow cells, the human cord blood, or any combination thereof can comprise CD34⁺ cells. A positive toxicity can be assigned if there is an increase in the biomarker level in the administered-to rodent compared to the non-administered-to rodent. A positive toxicity can be assigned if there is a decrease in the biomarker level in the administered-to rodent compared to the non-administered-to rodent.

In the method of testing a toxicity, the measuring can include measuring the biomarker level in the rodent at a first time point and a second time point, and the determining includes comparing the biomarker level measured at the second time point relative to the first time point. The first time point can be before, concurrent to, or after the administration of the therapy and the second time point can be after the administration of the therapy. The first time point and the second time point can both be after the administration of the therapy. The first time point occurs before the second time point. A positive toxicity can be assigned if there is an increase in the biomarker level at the second time point compared to the first time point. A positive toxicity can be assigned if there is a decrease in the biomarker level compared at the second time point compared to the first time point. In the method of testing a toxicity, the determining can include comparing the measured biomarker level to a predetermined threshold toxicity level. A positive toxicity can be assigned if the measured biomarker level is greater than or equal to the predetermined threshold toxicity level. A positive toxicity can be assigned if the measured biomarker level is less than or equal to the predetermined threshold toxicity level.

In the method of testing a toxicity, the biomarker can include, for example, a white blood cell count. Any type or combination of types of white blood cell can be counted. A decrease in white blood cells can indicate a positive toxicity of the therapy tested. An increase in white blood cells can indicate a positive toxicity of the therapy tested. Whether an increase or decrease corresponds to a positive toxicity can be depend on the type of white blood cell, the type of therapy tested, and/or other factors. Neutropenia can be measured. The biomarker can include, for example, rodent weight. A decrease in rodent weight over time can indicate a positive toxicity for the therapy tested.

The toxicity test can be performed on any rodent containing at least human hepatocytes and at least one of human hematopoietic bone marrow cells and human cord blood is provided by the present invention. The human hepatocytes, the human hematopoietic bone marrow cells, the human cord blood, or any combination thereof can comprise CD34⁺ cells. The rodent can further contain one or more types of human intestinal enzymes. The toxicity test can be performed on a mouse. The toxicity test can be performed on an immunodeficient rodent. The therapy tested can be targeted against any type of disease or disorder, or combination thereof For example, the therapy can, be an anti-cancer therapy. More than one therapy can be tested concurrently. Therapeutic compounds can be antagonists, agonists, or a combination thereof. The therapy can include the administration of one or more therapeutic compounds or compositions, for example, a small molecule, a nucleic acid, a protein, an antibody, or any combination thereof. For example, the compound can be a camptothecin, or an analog thereof of, such as topotecan (HYCAMTIN), irinotecan (CPT-11, CAMPTOSAR), DB 67 (AR67), BNP 1350, exatecan, lurtotecan, ST 1481, CKD 602, or a combination thereof Camptothecins are described in Giovanella et al., Science 246: 1046-1048, (1989). Various compounds are described, for instance, in U.S. Pat. Nos. 7,928,235, 7,572,803, 6,703,399, 6,699,875, 6,624,170, 6,407,239, and 6,407,118.

A method of testing the efficacy of a therapy is provided by the present invention. A therapy is administered to a rodent, the rodent containing at least human hepatocytes and at least one of human hematopoietic bone marrow cells and human cord blood. At least one efficacy metric in the rodent is measured. The efficacy of the therapy is determined based on the at least one efficacy metric measured. The efficacy of the therapy is assigned.

In the method of testing efficacy, the measuring can include measuring a biomarker level. The measuring can include measuring the biomarker level in the rodent at a first time point and a second time point, and the determining can include comparing the biomarker level measured at the second time point relative to the biomarker level measured at the first time point. The first time point can be before or concurrent to the administration of the therapy and the second time point is after the administration of the therapy. The first time point and the second time point can be after the administration of the therapy. The first time point occurs before the second time point. A positive efficacy can be assigned if there is an increase in the biomarker level at the second time point compared to the first time point. A positive efficacy can be assigned if there is a decrease in the biomarker level compared at the second time point compared to the first time point.

In the method of testing efficacy, the determining can include comparing the measured biomarker level to a predetermined threshold efficacy level. A positive efficacy can be assigned if the measured biomarker level is greater than or equal to the predetermined threshold efficacy level. A positive efficacy can be assigned if the measured biomarker level is less than or equal to the predetermined threshold efficacy level. The therapy tested can be targeted against any disease or disorder, or combination thereof, for example, an anti-cancer therapy. More than one therapy can be tested concurrently. The presence or absence of synergistic efficacy of two or more therapies can be tested. The therapy can include the administration of one or more therapeutic compounds or compositions, for example, a small molecule, a nucleic acid, a protein, an antibody, or any combination thereof. The biomarker can include a cancer cell marker.

In the method of testing efficacy, the measuring can include measuring tumor size and/or number. The measuring can include measuring tumor size and/or number at a first time point and a second time point, comparing tumor size and/or number measured at the first time point relative to that measured at the first time point, and the assigning includes assigning a positive efficacy if tumor size and/or number decreases. Measuring can include detecting the presence or absence of metastases. The method can include transplanting human tumor(s) into the rodent, before, during, or after the transplanting of the human hematopoietic bone marrow cells and/or the human hepatocytes.

The efficacy test can be performed on any rodent containing at least human hepatocytes and at least one of human hematopoietic bone marrow cells and human cord blood is provided by the present invention. The human hepatocytes, the human hematopoietic bone marrow cells, the human cord blood, or any combination thereof can comprise CD34⁺ cells. The rodent can further contain one or more types of human intestinal enzymes. The efficacy test can be performed on a mouse. The efficacy test can be performed on an immunodeficient rodent.

### EXAMPLES

### Example 1

For obtaining a double (liver and bone marrow) humanized mouse, a NSG mouse available from The Jackson Laboratory is used as a starting point. 8 to 12 weeks old NSG female mice are treated with 25 mg/kg busulfan ip 24 and 48 hours (or up to a week) prior to inoculation of bone marrow or cord blood cells to maximize engraftment. Bone marrow, e.g., human bone marrow samples, enriched to contain large amounts of stem cells, available from Reachbio of Seattle, Washington, are injected intravenously into the lateral tail vein of the mouse. Alternatively, or in addition, human CD34^{+/+} cord blood cells, available from ReachBio, can be transplanted, e.g., by transfusion. The progression of the engraftment is followed by blood smears obtained from the tail and stained with fluorescent antibodies specific for mouse and human cells, available from Calbiochem of San Diego, Calif.

Once the engraftment reaches its maximum and plateaus, the mice are anesthetized by gas anesthesia, laparotomized, and have an artificial liver inserted in the peritoneal cavity. The artificial liver is prepared using human frozen hepatocytes, available from CELSIS of Chicago, Illinois, suspended in a polymer solution, which is jelled in a mold resembling a human contact lens of about 1.5 cm diameter and maximum 2.5 mm thickness (1 2 mm is the maximum diffusion coefficient for oxygen and nutrients by imbibition). Construction of an artificial liver is described in Chen et al., Proc. Natl. Acad. Sci. U.S.A., 108 (29):11842-11847, 2011. A partial (two-third) hepatectomy is performed, for example as described in Nikfarjam et al., J. Investigative Surgery, 17 (5):291-294, 2004; Greene et al., J. Investigative Surgery, 16 (2):99-102, 2003; Zhang et al., J. Investigative Surgery, 23 (4):224-227, 2010. A suspension of human normal hepatocytes (1-2×10⁶ cells suspended in 200-300 μl) is inoculated under pressure in the spleen distal pole, which is then cauterized. The muscle layer is sutured with silk and the skin wound with steel grips. The mice are kept under infrared heating during recovery and under antibiotic and analgesic coverage (in the drinking water) and fed a whole wheat, whole milk paste for a week after the operation. Liver engraftment is monitored by measuring the level of Human Serum Albumin (HSA) and mouse serum albumin (MSA) in the blood by ELISA of serum samples obtained at regular intervals from the tail.

### Example 2

NSG mice obtained from Jackson Laboratory (8-12 weeks old females) are treated with 25 mg/kg 1P Busulfan 24 and 48 hours prior to inoculation of cord blood human hematopoietic cells enriched in stem cells, available from ReachBio, by IV (intravenously) in the lateral tail vein of the mouse. The progression of the engraftment is followed by blood smears obtained from the tail stained with fluorescent antibodies specific for mouse and human cells available from Cal Biochem of San Diego, Calif. Once the engraftment reaches its maximum and plateaus (6-12 weeks after the injection of the cord blood cells—depending on the size of the inoculum) the mice are anesthetized by gas anesthesia, laparotomized and from about two thirds to three quarters of the liver removed. A suspension containing human hepatocytes (1-2×10⁶ cells in complete medium is injected under pressure in the inferior pole of the spleen, which is then cauterized. The muscle layer is sutured with silk and the skin wound with steel grips. The mice are kept under infrared heating during recovery and under antibiotic and analgesic coverage (for example, in the drinking water) and fed a whole wheat bread soaked in milk, or other suitable diet, for a week after the operation. Liver engraftment is monitored by measuring the level of human serum albumin (HSA) in the blood by ELISA of serum samples, obtained at regular intervals from the tail.

### Example 3

Once maximum engraftment is reached as measured in Example 1 or 2, initial validation experiments are performed. A reasonable number of doubly humanized mice are obtained within approximately 5 to 6 months from the start of the humanizing process, i.e., 10-12 NSG mice with a >80% human BM and a >60% HSA in their blood. Such animals can demonstrate differences in their response to anticancer drugs. Camptothecin (CPT) is administered by gavage to humanized and non-humanized mice. At normal pH, CPT exists in equilibrium between two states: lactone and carboxylate. In the mouse, equilibrium is around 50% for each isoform. However, in human blood, due to an affinity for human serum albumin (HSA), the equilibrium moves towards the carboxylate form, leaving only 3%-5% total CPT in the active lactone form, see Giovanella et al., Annals New York Academy of Sciences 922: 27-35, 2000. The double humanized mouse can act akin to a human in this respect. Higher and higher doses of CPT are administered starting with a dose of 0.5 mg/kg orally by gavage given daily for 5 days followed by 2 days of rest for 3 weeks. Animals are weighed twice a week and their weight recorded. Blood is taken twice a week and blood cells counted. If no toxicity is found, the dose is raised by 50% and the procedure repeated until toxicity is obtained. Higher toxicity of CPT for the humanized HSG mice is found and the hematological toxicity of CPT is directly proportional to the percentage of human bone marrow. Present in the mouse, human bone marrow cells are more sensitive to camptothecin than mouse cells.

### Example 4

Once a maximum tolerated dose (MTD) for humanized NSG mice and MTD for non-humanized ones is obtained using the procedure described in Example 3, final validation experiments are performed. Six human tumors are xenotransplanted subcutaneously on the dorsal flank in humanized and non-humanized NSG mice. Once the tumors become palpable, the animals are treated with the MTD dose of CPT of their group, i.e., the humanized mice with the humanized MTD and the non-humanized mice with their MTD. The tumors are measured with calipers twice a week and the results recorded. A much higher anticancer activity of CPT in the non-humanized group can be found as reflected in the relative size of the tumors in the two groups, higher anticancer activity being associated with decreased tumor size. A human dose for CPT can be determined and suitability of the drug for treatment of humans can be determined. The experiments can be performed substituting one or more drugs in place of CPT. The experiments can be performed using one or more drugs in addition to CPT. The experiments can be performed by using introduction of human leukemia cancer cells or other non-solid tumor human cancer cells into the rodent instead of xenotransplantation of solid tumors. Such cancers can be evaluated based on alternative metrics such as cell count.

### Example 5

The procedures in any one or combination of Examples 1-4 are performed with mice having a higher percentage of human bone marrow and hepatocytes. Rather than having a non-human control, the control can be instead, or in addition, mice having the percentage of human bone marrow (and/or human cord blood) used in the experimental group in the first performance of the procedures. Double humanized mice are further supplemented with human or humanized intestinal enzymes to form a triply humanized mouse (bone marrow, liver, and intestinal enzymes), which is then be used as a predictor of human response to anticancer agents. A mouse having humanized intestinal enzymes can be constructed, for example, as described in van Herwaarden et al., J. Clin. Invest., 117 (11):3583-3592, 2007. A higher anticancer activity of CPT in the less humanized group can be found as reflected in the relative size of the tumors in the two groups, higher anticancer activity being associated with decreased tumor size. A human dose for CPT can be redetermined and suitability of the drug for treatment of humans can be redetermined.

The present invention includes the following aspects/embodiments/features in any order and/or in any combination:

1. A rodent comprising at least human hepatocytes and at least one of human hematopoietic bone marrow cells and human cord blood.

2. The rodent of any preceding or following embodiment/feature/aspect, comprising human hepatocytes and human hematopoietic bone marrow cells.

3. The rodent of any preceding or following embodiment/feature/aspect, wherein human hepatocytes, the human hematopoietic bone marrow cells, the human cord blood, or any combination thereof comprise CD34+ cells.

4. The rodent of any preceding or following embodiment/feature/aspect, wherein the rodent comprises partially humanized bone marrow and a partially humanized liver.

5. The rodent of any preceding or following embodiment/feature/aspect, wherein the rodent comprises fully humanized bone marrow and a partially humanized liver.

6. The rodent of any preceding or following embodiment/feature/aspect, wherein the rodent comprises an artificial liver comprising human hepatocytes.

7. The rodent of any preceding or following embodiment/feature/aspect, wherein the rodent is immunodeficient.

8. The rodent of any preceding or following embodiment/feature/aspect, wherein the rodent is severely combined immunodeficient.

9. The rodent of any preceding or following embodiment/feature/aspect, wherein the rodent is a mouse.

10. The rodent of any preceding or following embodiment/feature/aspect, wherein the mouse is a NOD/SCID/1L2ZY null (NSG) mouse.

11. The rodent of any preceding or following embodiment/feature/aspect, wherein the rodent comprises human neoplastic cells.

12. The rodent of any preceding or following embodiment/feature/aspect, wherein the rodent comprises a human tumor.

13. The rodent of any preceding or following embodiment/feature/aspect, wherein the human tumor comprises a human carcinoma, a human sarcoma, a human lymphoma, or a combination thereof.

14. The rodent of any preceding or following embodiment/feature/aspect, wherein the rodent is female.

15. The rodent of any preceding or following embodiment/feature/aspect, further comprising human intestinal enzymes.

16. A method of producing a rodent comprising at least human hepatocytes and at least one of human hematopoietic bone marrow cells and human cord blood, the method comprising:
preparing a rodent for bone marrow engraftment;
transplanting at least one of human hematopoietic bone marrow cells and human cord blood into the rodent; and
transplanting human hepatocytes into the rodent.

17. The method of any preceding or following embodiment/feature/aspect, comprising transplanting human hematopoietic bone marrow cells into the rodent.

18. The method of any preceding or following embodiment/feature/aspect, wherein the human hepatocytes, the human hematopoietic bone marrow cells, the human cord blood, or any combination thereof comprise CD34⁺ cells.

19. The method of any preceding or following embodiment/feature/aspect, wherein the preparation comprises administering busulfan or a pharmaceutically acceptable salt thereof to the rodent.

20. The method of any preceding or following embodiment/feature/aspect, wherein the transplanting of human bone marrow cells comprises transplanting human hematopoietic bone marrow cells enriched in human stem cells.

21. The method of any preceding or following embodiment/feature/aspect, wherein the transplanting of human hematopoietic bone marrow cells comprises injecting human hematopoietic bone marrow cells into the rodent.

22. The method of any preceding or following embodiment/feature/aspect, wherein the transplanting of human hepatocytes comprises transplanting an artificial liver comprising human hepatocytes into the peritoneal cavity of the rodent.

23. The method of any preceding or following embodiment/feature/aspect, wherein the transplanting of human hepatocytes comprises inoculating human hepatocytes into the spleen of the rodent.

24. The method of any preceding or following embodiment/feature/aspect, further comprising performing a partial or full hepatectomy on the rodent.

25. The method of any preceding or following embodiment/feature/aspect, further comprising transplanting a human neoplastic cell into the rodent.

26. The method of any preceding or following embodiment/feature/aspect, further comprising transplanting a human tumor into the rodent.

27. The method of any preceding or following embodiment/feature/aspect, wherein the human tumor comprises a human carcinoma, a human sarcoma, a human lymphoma, or a combination thereof.

28. The method of any preceding or following embodiment/feature/aspect, wherein the rodent is female.

29. The method of any preceding or following embodiment/feature/aspect, wherein the rodent is immunodeficient.

30. The method of any preceding or following embodiment/feature/aspect, wherein the rodent is a mouse.

31. The method of any preceding or following embodiment/feature/aspect, wherein the mouse is a NOD/SCID/1L2ZY null (NSG) mouse.

32. The method of any preceding or following embodiment/feature/aspect, further comprising transplanting a human intestinal enzyme, a cell expressing a human intestinal enzyme, genetic material encoding a human intestinal enzyme, or a combination thereof, into the rodent.

33. A method of testing a toxicity of a therapy comprising administering a therapy to a rodent, the rodent comprising at least human hepatocytes and at least one of human hematopoietic bone marrow cells and human cord blood;
measuring at least one toxicity metric in the rodent;
determining the toxicity of the therapy based on the at least one toxicity metric measured; and
assigning the toxicity based on the determination.

34. The method of any preceding or following embodiment/feature/aspect, wherein the rodent comprises human hepatocytes and human hematopoietic bone marrow cells.

35. The method of any preceding or following embodiment/feature/aspect, wherein the human hepatocytes, the human hematopoietic bone marrow cells, the human cord blood, or any combination thereof comprise CD34⁺ cells.

36. The method of any preceding or following embodiment/feature/aspect, wherein the measuring comprises measuring a biomarker level in the rodent.

37. The method of any preceding or following embodiment/feature/aspect, wherein the measuring further comprises measuring the biomarker level in a rodent that is not administered the therapy, and the determining comprises comparing the measured biomarker level in the administered-to rodent relative to the measured biomarker level in the non-administered-to rodent.

38. The method of any preceding or following embodiment/feature/aspect, wherein the non-administered-to rodent comprises at least human hepatocytes and at least one of human hematopoietic bone marrow cells and human cord blood.

39. The method of any preceding or following embodiment/feature/aspect, wherein the assigning comprises assigning a positive toxicity if there is an increase in the biomarker level in the administered-to rodent compared to the non-administered-to rodent.

40. The method of any preceding or following embodiment/feature/aspect, wherein the assigning comprises assigning a positive toxicity if there is a decrease in the biomarker level in the administered-to rodent compared to the non-administered-to rodent.

41. The method of any preceding or following embodiment/feature/aspect, wherein the measuring comprises measuring the biomarker level in the rodent at a first time point and a second time point, and the determining comprises comparing the biomarker level measured at the second time point relative to the first time point.

42. The method of any preceding or following embodiment/feature/aspect, wherein the first time point is before or concurrent to the administration of the therapy and the second time point is after the administration of the therapy.

43. The method of any preceding or following embodiment/feature/aspect, wherein the first time point and the second time point are after the administration of the therapy.

44. The method of any preceding or following embodiment/feature/aspect, wherein the assigning comprises assigning a positive toxicity if there is an increase in the biomarker level at the second time point compared to the first time point.

45. The method of any preceding or following embodiment/feature/aspect, wherein the assigning comprises assigning a positive toxicity if there is a decrease in the biomarker level compared at the second time point compared to the first time point.

46. The method of any preceding or following embodiment/feature/aspect, wherein the determining comprises comparing the measured biomarker level to a predetermined threshold toxicity level.

47. The method of any preceding or following embodiment/feature/aspect, wherein the assigning comprises assigning a positive toxicity if the measured biomarker level is greater than or equal to the predetermined threshold toxicity level.

48. The method of any preceding or following embodiment/feature/aspect, wherein the assigning comprises assigning a positive toxicity if the measured biomarker level is less than or equal to the predetermined threshold toxicity level.

49. The method of any preceding or following embodiment/feature/aspect, wherein the biomarker comprises a white blood cell count.

50. The method of any preceding or following embodiment/feature/aspect, wherein the biomarker comprises rodent weight.

51. The method of any preceding or following embodiment/feature/aspect, wherein the rodent is a mouse.

52. The method of any preceding or following embodiment/feature/aspect, wherein the rodent is immunodeficient.

53. The method of any preceding or following embodiment/feature/aspect, wherein the rodent further comprises human intestinal enzymes.

54. The method of any preceding or following embodiment/feature/aspect, wherein the therapy comprises an anti-cancer therapy.

55. A method of testing the efficacy of a therapy comprising:
administering a therapy to a rodent, the rodent comprising at least human hepatocytes and at least one of human hematopoietic bone marrow cells and human cord blood;
measuring at least one efficacy metric in the rodent;
determining the efficacy of the therapy based on the at least one efficacy metric measured; and
assigning the efficacy of the therapy.

56. The method of any preceding or following embodiment/feature/aspect, wherein the rodent comprises human hepatocytes and human hematopoietic bone marrow cells.

57. The method of any preceding or following embodiment/feature/aspect, wherein the human hepatocytes, the human hematopoietic bone marrow cells, the human cord blood, or any combination thereof comprise CD34⁺ cells.

58. The method of any preceding or following embodiment/feature/aspect, wherein the measuring comprises measuring a biomarker level.

59. The method of any preceding or following embodiment/feature/aspect, wherein the measuring comprises measuring the biomarker level in the rodent at a first time point and a second time point, and the determining comprises comparing the biomarker level measured at the second time point relative to the biomarker level measured at the first time point.

60. The method of any preceding or following embodiment/feature/aspect, wherein the first time point is before or concurrent to the administration of the therapy and the second time point is after the administration of the therapy.

61. The method of any preceding or following embodiment/feature/aspect, wherein the first time point and the second time point are after the administration of the therapy.

62. The method of any preceding or following embodiment/feature/aspect, wherein the assigning comprises assigning a positive efficacy if there is an increase in the biomarker level at the second time point compared to the first time point.

63. The method of any preceding or following embodiment/feature/aspect, wherein the assigning comprises assigning a positive efficacy if there is a decrease in the biomarker level compared at the second time point compared to the first time point.

64. The method of any preceding or following embodiment/feature/aspect, wherein the determining comprises comparing the measured biomarker level to a predetermined threshold efficacy level.

65. The method of any preceding or following embodiment/feature/aspect, wherein the assigning comprises assigning a positive efficacy if the measured biomarker level is greater than or equal to the predetermined threshold to the efficacy level.

66. The method of any preceding or following embodiment/feature/aspect, wherein the assigning comprises assigning a positive efficacy if the measured biomarker level is less than or equal to the predetermined threshold efficacy level.

67. The method of any preceding or following embodiment/feature/aspect, wherein the therapy comprises an anti-cancer therapy.

68. The method of any preceding or following embodiment/feature/aspect, wherein the biomarker is a cancer cell marker.

69. The method of any preceding or following embodiment/feature/aspect, wherein the measuring comprises measuring at least one of tumor size and number.

70. The method of any preceding or following embodiment/feature/aspect, wherein the measuring comprises measuring at least of one tumor size and number at a first time point and a second time point, comparing at least one of tumor size and number measured at the first time point relative to that measured at the first time point, and the assigning comprises assigning a positive efficacy if at least one of tumor size and number decreases.

71. The method of any preceding or following embodiment/feature/aspect, further comprising transplanting at least one human tumor into the rodent.

72. The method of any preceding or following embodiment/feature/aspect, wherein the rodent is a mouse.

73. The method of any preceding or following embodiment/feature/aspect, wherein the rodent is immunodeficient.

74. The method of any preceding or following embodiment/feature/aspect, wherein the rodent further comprises human intestinal enzymes.

The present invention can include any combination of these various features or embodiments above and/or below as set forth in sentences and/or paragraphs. Any combination of disclosed features herein is considered part of the present invention and no limitation is intended with respect to combinable features.

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

Other embodiments of the present invention will be apparent to those skilled in the art from consideration of the present specification and practice of the present invention disclosed herein. It is intended that the present specification and examples be considered as exemplary only with a true scope and spirit of the invention being indicated by the following claims and equivalents thereof.

What is claimed is:

1. A rodent comprising at least human hepatocytes and at least one of human hematopoietic bone marrow cells and human cord blood.

2. The rodent of claim 1 comprising human hepatocytes and human hematopoietic bone marrow cells.

3. The rodent of claim 1, wherein human hepatocytes, the human hematopoietic bone marrow cells, the human cord blood, or any combination thereof comprise CD34+ cells.

4. The rodent of claim 1, wherein the rodent comprises partially humanized bone marrow and a partially humanized liver.

5. The rodent of claim 1, wherein the rodent comprises fully humanized bone marrow and a partially humanized liver.

6. The rodent of claim 1, wherein the rodent comprises an artificial liver comprising human hepatocytes.

7. The rodent of claim 1, wherein the rodent is immunodeficient.

8. The rodent of claim 1, wherein the rodent is severely combined immunodeficient.

9. The rodent of claim 1, wherein the rodent is a mouse.

10. The rodent of claim 9, wherein the mouse is a NOD/SCID/1L2ZY null (NSG) mouse.

11. The rodent of claim 1, wherein the rodent comprises human neoplastic cells.

12. The rodent of claim 1, wherein the rodent comprises a human tumor.

13. The rodent of claim 12, wherein the human tumor comprises a human carcinoma, a human sarcoma, a human lymphoma, or a combination thereof.

14. The rodent of claim 1, wherein the rodent is female.

15. The rodent of claim 1, further comprising human intestinal enzymes.

16. A method of producing a rodent comprising at least human hepatocytes and at least one of human hematopoietic bone marrow cells and human cord blood, the method comprising:
preparing a rodent for bone marrow engraftment;
transplanting at least one of human hematopoietic bone marrow cells and human cord, blood into the rodent; and
transplanting human hepatocytes into the rodent.

17. The method of claim 16, comprising transplanting human hematopoietic bone marrow cells into the rodent.

18. The method of claim 16, wherein the human hepatocytes, the human hematopoietic bone marrow cells, the human cord blood, or any combination thereof comprise CD34⁺ cells.

19. The method of claim 16, wherein the preparation comprises administering busulfan or a pharmaceutically acceptable salt thereof to the rodent.

20. The method of claim 16, wherein the transplanting of human bone marrow cells comprises transplanting human hematopoietic bone marrow cells enriched in human stem cells.

21. The method of claim 16, wherein the transplanting of human hematopoietic bone marrow cells comprises injecting human hematopoietic bone marrow cells into the rodent.

22. The method of claim 16, wherein the transplanting of human hepatocytes comprises transplanting an artificial liver comprising human hepatocytes into the peritoneal cavity of the rodent.

23. The method of claim 16, wherein the transplanting of human hepatocytes comprises inoculating human hepatocytes into the spleen of the rodent.

24. The method of claim 16, further comprising performing a partial or full hepatectomy on the rodent.

25. The method of claim 16, further comprising transplanting a human neoplastic cell into the rodent.

26. The method of claim 16, further comprising transplanting a human tumor into the rodent.

27. The method of claim 26, wherein the human tumor comprises a human carcinoma, a human sarcoma, a human lymphoma, or a combination thereof

28. The method of claim 16, wherein the rodent is female.

29. The method of claim 16, wherein the rodent is immunodeficient.

30. The method of claim 16, wherein the rodent is a mouse.

31. The method of claim 30, wherein the mouse is a NOD/SCID/1L2ZY null (NSG) mouse.

32. The method of claim 16, further comprising transplanting a human intestinal enzyme, a cell expressing a human intestinal enzyme, genetic material encoding a human intestinal enzyme, or a combination thereof, into the rodent.

33. A method of testing a toxicity of a therapy comprising
administering a therapy to a rodent, the rodent comprising at least human hepatocytes and at least one of human hematopoietic bone marrow cells and human cord blood;
measuring at least one toxicity metric in the rodent;
determining the toxicity of the therapy based on the at least one toxicity metric measured; and
assigning the toxicity based on the determination.

34. The method of claim 33, wherein the rodent comprises human hepatocytes and human hematopoietic bone marrow cells.

35. The method of claim 33, wherein the human hepatocytes, the human hematopoietic bone marrow cells, the human cord blood, or any combination thereof comprise CD34⁺ cells.

36. The method of claim 33, wherein the measuring comprises measuring a biomarker level in the rodent.

37. The method of claim 36, wherein the measuring further comprises measuring the biomarker level in a rodent that is not administered the therapy, and the determining comprises comparing the measured biomarker level in the administered-to rodent relative to the measured biomarker level in the non-administered-to rodent.

38. The method of claim 37, wherein the non-administered-to rodent comprises at least human hepatocytes and at least one of human hematopoietic bone marrow cells and human cord blood.

39. The method of claim 37, wherein the assigning comprises assigning a positive toxicity if there is an increase in the biomarker level in the administered-to rodent compared to the non-administered-to rodent.

40. The method of claim 37, wherein the assigning comprises assigning a positive toxicity if there is a decrease in the biomarker level in the administered-to rodent compared to the non-administered-to rodent.

41. The method of claim 36, wherein the measuring comprises measuring the biomarker level in the rodent at a first time point and a second time point, and the determining comprises comparing the biomarker level measured at the second time point relative to the first time point.

42. The method of claim 41, wherein the first time point is before or concurrent to the administration of the therapy and the second time point is after the administration of the therapy.

43. The method of claim 41, wherein the first time point and the second time point are after the administration of the therapy.

44. The method of claim 41, wherein the assigning comprises assigning a positive toxicity if there is an increase in the biomarker level at the second time point compared to the first time point.

45. The method of claim 41, wherein the assigning comprises assigning a positive toxicity if there is a decrease in the biomarker level compared at the second time point compared to the first time point.

46. The method of claim 36, wherein the determining comprises comparing the measured biomarker level to a predetermined threshold toxicity level.

47. The method of claim 46, wherein the assigning comprises assigning a positive toxicity if the measured biomarker level is greater than or equal to the predetermined threshold toxicity level.

48. The method of claim 46, wherein the assigning comprises assigning a positive toxicity if the measured biomarker level is less than or equal to the predetermined threshold toxicity level.

49. The method of claim 36, wherein the biomarker comprises a white blood cell count.

50. The method of claim 36, wherein the biomarker comprises rodent weight.

51. The method of claim 33, wherein the rodent is a mouse.

52. The method of claim 33, wherein the rodent is immunodeficient.

53. The method of claim 33, wherein the rodent further comprises human intestinal enzymes.

54. The method of claim 33, wherein the therapy comprises an anti-cancer therapy.

55. A method of testing the efficacy of a therapy comprising:
administering a therapy to a rodent, the rodent comprising at least human hepatocytes and at least one of human hematopoietic bone marrow cells and human cord blood;
measuring at least one efficacy metric in the rodent;
determining the efficacy of the therapy based on the at least one efficacy metric measured; and
assigning the efficacy of the therapy.

56. The method of claim 55, wherein the rodent comprises human hepatocytes and human hematopoietic bone marrow cells.

57. The method of claim 55, wherein the human hepatocytes, the human hematopoietic bone marrow cells, the human cord blood, or any combination thereof comprise CD34⁺ cells.

58. The method of claim 55, wherein the measuring comprises measuring a biomarker level.

59. The method of claim 58, wherein the measuring comprises measuring the biomarker level in the rodent at a first time point and a second time point, and the determining comprises comparing the biomarker level measured at the second time point relative to the biomarker level measured at the first time point.

60. The method of claim 59, wherein the first time point is before or concurrent to the administration of the therapy and the second time point is after the administration of the therapy.

61. The method of claim 59, wherein the first time point and the second time point are after the administration of the therapy.

62. The method of claim 59, wherein the assigning comprises assigning a positive efficacy if there is an increase in the biomarker level at the second time point compared to the first time point.

63. The method of claim 59, wherein the assigning comprises assigning a positive efficacy if there is a decrease in the biomarker level compared at the second time point compared to the first time point.

64. The method of claim 58, wherein the determining comprises comparing the measured biomarker level to a predetermined threshold efficacy level.

65. The method of claim 64, wherein the assigning comprises assigning a positive efficacy if the measured biomarker level is greater than or equal to the predetermined threshold to the efficacy level.

66. The method of claim 64, wherein the assigning comprises assigning a positive efficacy if the measured biomarker level is less than or equal to the predetermined threshold efficacy level.

67. The method of claim 55, wherein the therapy comprises an anti-cancer therapy.

68. The method of claim 58, wherein the biomarker is a cancer cell marker.

69. The method of claim 55, wherein the measuring comprises measuring at least one of tumor size and number.

70. The method of claim 69, wherein the measuring comprises measuring at least of one tumor size and number at a first time point and a second time point, comparing at least one of tumor size and number measured at the first time point relative to that measured at the first time point, and the assigning comprises assigning a positive efficacy if at least one of tumor size and number decreases.

71. The method of claim 69, further comprising transplanting at least one human tumor into the rodent.

72. The method of claim 55, wherein the rodent is a mouse.

73. The method of claim 55, wherein the rodent is immunodeficient.

74. The method of claim 55, wherein the rodent further comprises human intestinal enzymes.
